(19) United States
(12) Patent Application Publication
(10) Pub. No.: US 2009/0319026 A1
(43) Pub. Date: Dec. 24, 2009

(54) Composite Stent with Reservoirs for Drug Delivery and Methods of Manufacturing

(75) Inventor: Michael P. Meyer, Richfield, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 12/143,189

(22) Filed: Jun. 20, 2008

(51) Int. Cl. A61F 2/82; B26D 7/14; B29C 53/08

(52) U.S. Cl. 623/1.16; 623/1.42; 83/17; 156/218

(57) ABSTRACT

A composite stent structure and methods of manufacturing same, include a stent wall having two or more layers of the same or different material. The wall provides various means of drug delivery while maintaining the stent's overall structure. The outer layers and/or inner layers may have holes, reservoirs, or openings that allow for the delivery of drugs or other therapeutic agents.

### CROSS-REFERENCE TO RELATED APPLICATIONS

Not Applicable

### STATEMENT REGARDING FEDERALLY SPONSORED RESEARCH

Not Applicable

### FIELD OF THE INVENTION

In some embodiments this invention relates to implantable medical devices, materials used for such devices, and their manufacture. Some embodiments of the invention are directed more specifically to stents used to deliver drugs and other beneficial agents into a bodily lumen.

### BACKGROUND OF THE INVENTION

The use of stents in bodily lumen is well known. A stent is typically delivered in an unexpanded state to a desired location in a bodily lumen via a stent delivery device such as a catheter. Once the stent is at the desired bodily location, it is either expanded with a balloon or other suitable device or allowed to expand, for example, by withdrawing a restraining sheath. Because the stent needs to, in some way, be expanded at the desired location, the stent structure must be flexible.

Typically stents are constructed using either a solid wire member or a thin-walled tubular member made of polymers, organic fabrics, shape memory alloys (such as nitinol), or biocompatible metals (such as stainless steel, gold, silver, titanium, tantalum). In some designs, the stents are formed with such members that act as connectors and struts. These connectors and struts create a repeating or non-repeating pattern to allow for the expansion of the stent while also providing structural support.

In addition to providing structural support in the bodily lumen, stents have been used to supply a wide variety of treatments by delivering drugs and other beneficial agents to a desired bodily location. Such agents can be coated on the stent, or can be contained within the stent with holes for the drug to elute at the proper location.

While surface coatings provide an efficient means of manufacturing a stent to deliver an agent, they have several disadvantages. Surface coatings can provide very little control over the release of the drug into the lumen; the drugs might breakdown too easily, or not easily enough, depending on certain conditions. While the rapid breakdown of the surface coating can be improved by increasing the thickness of the surface coat, this increases the thickness of the stent as a whole, which can lead to increased trauma to the lumen during implantation, reduced flow rates, and increased vulnerability of the coating to damage or failure. In addition because the surface coating can erode, unwanted space may be created between the vessel wall and the stent, allowing for undesirable motion between the stent and the wall. In addition, many types of drugs and beneficial agents cannot currently be delivered using a surface coating, so treatment options with a surface coated stent are limited.

Another well-known means of drug delivery is to manufacture the stent with a plurality holes or other openings in the stent to allow for controlled release of the agent. The stent openings or holes are loaded with the desired beneficial agent, and the stent is implanted in the lumen. This design allows the stent to better achieve a desired agent delivery profile and allows the stent to deliver a relatively large volume of an agent, but also has its drawbacks. Unlike the surface coated stents, creating holes and other openings can negatively affect the structural integrity of the stent.

Accordingly, it would be desirable to have a drug delivery device that would provide an effective means of releasing the beneficial agent while also maintaining the structural integrity of the device.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well for the purposes of complying with 37 C.F.R. 1.72.

### BRIEF SUMMARY OF THE INVENTION

In at least one embodiment the present invention involves merging various materials to create a composite structure for a stent such that the stent can provide various means of drug delivery while maintaining its overall structure. The composite structure is made of two or more layers of materials fused, bonded, or joined in some way. The outer layers and/or inner layers may have holes, reservoirs, or openings that allow for the delivery of drugs.

In at least one embodiment, the stent structure has three layers where the center layer remains solid and holes, reservoirs or openings are made in the outer and/or inner layer of the stent for drug delivery. These reservoirs only extend to the solid inner layer so that the stent maintains its structure, while allowing the reservoir to be maximized in size. The layers can be of the same or different materials.

In at least one embodiment, the center layer can have a pattern such as checks, braids, or slots such that at least some of the openings in the outer layer effectively extend through the entire wall thickness of the stent. In some embodiments the stent structure is provided with two or more layers with holes, reservoirs, or openings through one, several or all of the layers.

In at least one embodiment the invention is directed to a method of manufacturing a composite stent. An example of one method comprises the use of laser, mechanical, water jet, etc. or other mechanisms to cut, drill, bore or otherwise form holes or reservoirs with at least one opening in the desired layer or layers and then join the layers into a single composite sheet. The sheet is later rolled and the ends welded together to form a tube of composite material from which a stent may be cut. Another method of manufacturing the composite stent is to coat, wrap or otherwise affix the material to a wire or other structural member. Once the structural member or wire is coated with the material, reservoirs could be cut into the material.

Additional details and/or embodiments of the invention are discussed below. These and other embodiments which characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there is illustrated and described embodiments of the invention.

### BRIEF DESCRIPTION OF DRAWINGS

A detailed description of the invention is hereafter described with specific reference being made to the drawings.

FIG. 1 shows a portion of an embodiment of the composite structure stent for drug delivery.

FIGS. 2a-2d show cross sections of the composite structure stent of four different embodiments.

FIGS. 3a-3d show a cross sectional views of the composite stent walls of four different embodiments.

FIG. 4 shows one method of manufacturing the composite stent.

FIG. 5 shows another method of manufacturing the composite stent.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

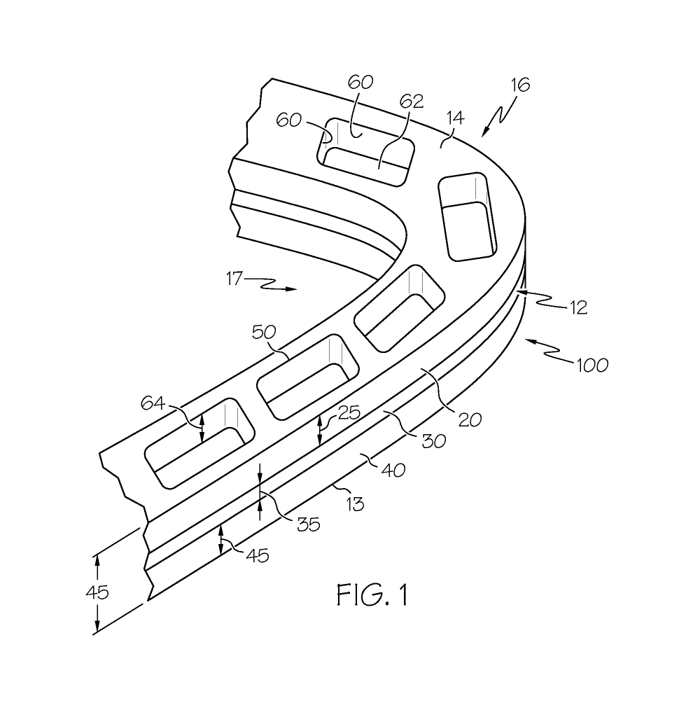

In one embodiment, the invention is directed to a stent such as that shown generally at 100 in FIG. 1. Stent 100 has a wall 12 that defines an inner stent surface 13 and an outer stent surface 14 and a thickness 15. Wall 12 has a plurality of interconnected stent members such as struts and connectors as shown generally at 16, and a plurality of stent openings such as those shown generally at 17. Wall 12 has three layers: a first layer 20, a support layer 30, and a second layer 40 (however, wall 12 can be comprised of two, three or more layers). Each layer 20, 30, 40 can be made from the same material or different materials. Materials used for these layers include but are not limited to polymers, organic fabrics, shape memory alloys (such as nitinol), or biocompatible metals (such as stainless steel, gold, silver, titanium, tantalum). It is preferred that first layer 20 and second layer 40 are made from a different material than support layer 30. The material used in first layer 20 or second layer 40 can be optimized in order to allow a drug or other beneficial agent to be effectively held, while the support layer 30 can be optimized for overall strength.

As such, a wide variety of materials are suitable for use in the construction of the wall 12. For example, in some embodiments the support layer 30 is constructed from any of a variety of metals including, but not limited to: stainless steel, titanium, cobalt, chromium, platinum, and/or alloys thereof (i.e. cobalt chromium, platinum chromium, etc.). In some embodiments layers 20 and 40 are constructed of any of a variety of biocompatible polymer materials. Examples of suitable polymer materials include but are not limited to: polyurethane, polyethylene (an/or blends thereof), polylactic acid (PLA), poly(lactic-co-glycolic acid) (PLGA), polycarbonate blends, etc.

In some embodiments one or both of the first and second layers 20 and 40 are constructed of a biocompatible metal similar or different than the material of the support layer 30.

Each layer 20, 30, 40 has a given thickness 25, 35, 45. Thicknesses 25, 35, 45 may or may not be equal (e.g. thickness 25 of first layer 20 may or may not be the same as thickness 45 of second layer 40, and thicknesses 25, 45 may or may not be the same as thickness 35 of support layer 30). Together, thicknesses 25, 35, 45 equal the overall thickness 15 of the wall 12.

Again referring to FIG. 1, first layer 20 has holes, reservoirs, or openings 50 of a certain depth 65 to allow for beneficial agents 70 to be loaded into the stent. Beneficial or therapeutic agent(s) 70 may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate. Some examples of therapeutic agents suitable for use in the reservoirs 50 of the present application are described in at least the following references the entire contents of each being incorporated by reference: U.S. Pat. No. 7,041,130 issued to Santini; U.S. Pat. No. 6,783,543 to Jang; U.S. Pat. No. 7,208,011 to Shanley; U.S. Pat. No. 7,056,338 to Shanley; and U.S. Patent Publication No. 2007005124 to De Scheerder.

The depth 65 of the reservoir 50 may or may not penetrate through the entire thickness 25 of first layer 20, and furthermore depth 65 may or may not penetrate through overall stent thickness 15. In other words, each reservoir 50 is not necessarily a throughhole, and depth 65 may or may not be equivalent to thickness 25 or overall stent thickness 15. Depth 65 is only limited by overall stent thickness 15 and is not limited by thickness 25, 35, 45 of layer 20, 30, 40.

In some embodiments, the sides 60 of the reservoirs 50 are defined by first layer 20 or second layer 40, and the bottom 62 of the reservoir 50 is defined by the support layer 30. In such embodiments, the support layer 30 can be of a material that is porous, or of a lose braid, or may be made of a pattern (including but not limited to checks, braids, or slots) such that openings 50 on opposing sides of the support layer 30 may have some degree of fluid communication, thereby allowing the opening 50 to act as a throughhole without compromising the structural integrity of the support layer 30 and the stent member 17.

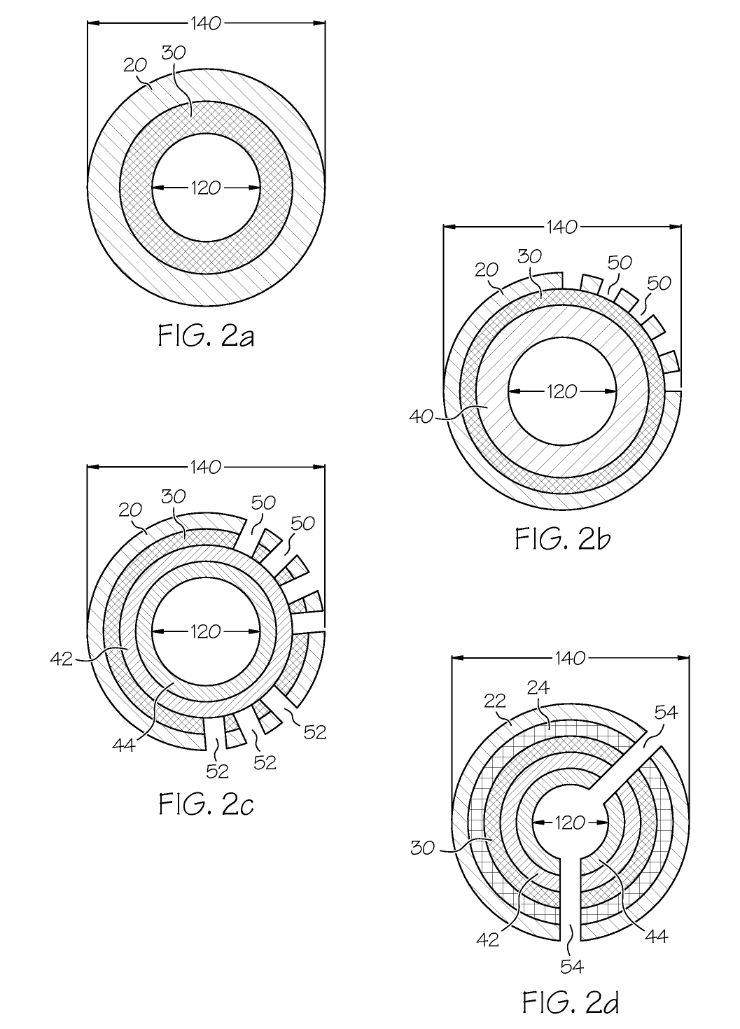

Referring now to FIGS. 2a-2d, first layer 20 can be a single layer of materials, or can be a plurality of layers of material 22, 24. These materials include but are not limited to metals, polymers, ceramics, shape memory alloys, and fabrics and can be used in a variety of combinations as shown. The use of a variety of materials and the creation of such a composite structure allows for optimization of the physical properties in the stent. Additionally, second layer 40 can be a single layer of materials or can be a plurality of layers of material 42, 44.

An opening in the stent 100 of inner diameter 120 must remain so that a balloon or other device can be used to expand the stent and also to allow for fluid flow in the vessel. In addition, inner diameter 120 can remain constant or may vary throughout stent 100. Outer diameter 140 also can remain constant or may vary throughout stent 100.

Still referring to FIGS. 2a-2d, reservoirs, holes and other openings 50 can be created in a variety of arrangements, on any of the layers, in many different combinations. As examples, reservoir 50 on FIG. 2b can go through first layer 20, or a reservoir 52 be molded into layers 20 and 30 as shown on FIG. 2c, or a hole 54 can be created through all layers 20, 30, 40 as shown on FIG. 2d. These and other opening arrangements can be created and/or combined with other opening arrangements to customize the stent based on the desired type of treatment to achieve optimal results.

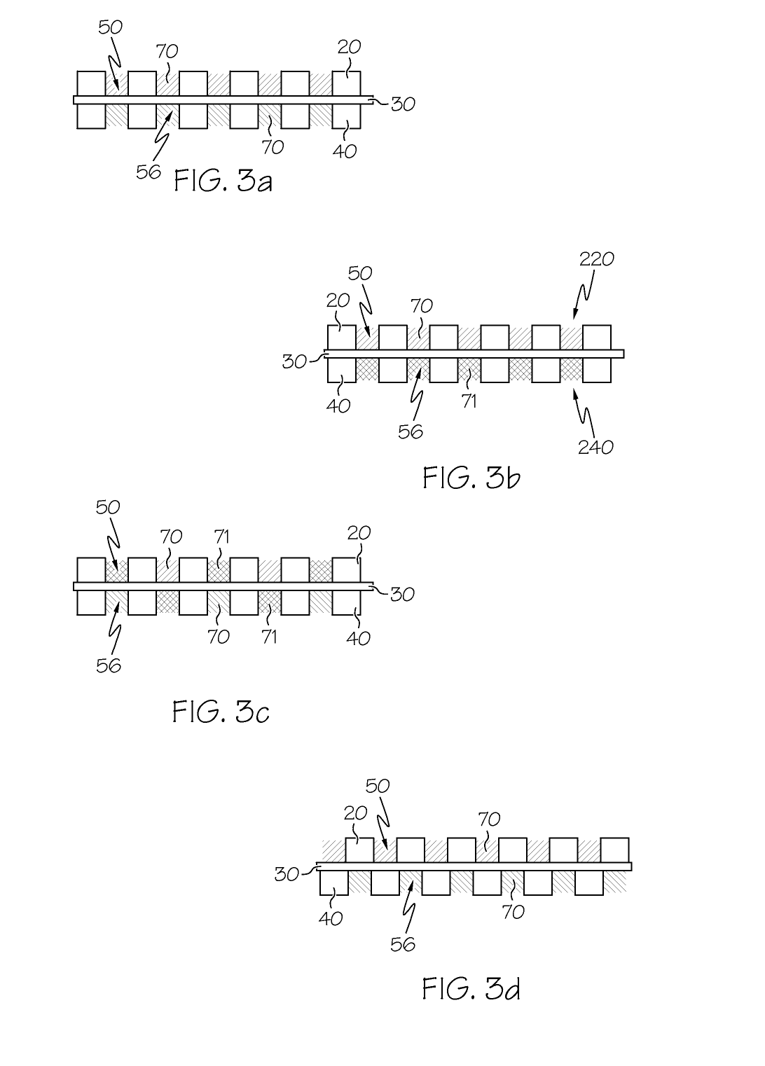

Referring to FIGS. 3a-3d, the reservoirs, holes and other openings 50, 56 created in layers 20 and 40 also can contain various combinations of drugs or beneficial agents 70, 71 for different treatment purposes. FIGS. 3a-3d show a first layer 20, a support layer 30, and a second layer 40. Support layer 30 in this embodiment remains solid, while reservoirs 50, 56 have been created in both the first layer 20 and the second layer 40. In FIG. 3a, reservoirs 50 and 56 both contain the same drug or beneficial agent 70. In FIG. 3b, since the openings 50, 56 do not extend all the way through the stent, alternative drugs, polymers, or other beneficial agents can be used on the luminal side 220 versus the abluminal diameter 240. FIG. 3b shows reservoirs 50 in the first layer 20 all contain the same drug or beneficial agent 70, while the reservoirs 56 in the second layer 40 contain all the same drug 71, but drug 70 is different than drug 71. FIG. 3c, as opposed to FIG. 3b, shows the reservoirs 50 in the first layer 20 containing different drugs 70, 71 in each reservoir 50. This alternating pattern allows for customized drug delivery that may be beneficial in certain applications. FIG. 3d shows an embodiment where reservoirs 50 are not parallel with reservoirs 56. In other words, the first layer reservoirs 50 are offset from the second layer reservoirs 56. This arrangement and other similar arrangements allow for added structural support and customized drug delivery. Other similar combinations and arrangements can be created in order to achieve the optimal drug delivery profile.

In some embodiments, the support layer, first layer, or second layer can be constructed of one or more wires or wire-like members.

In order to manufacture the composite structure and create the stent, several embodiments are presented below, but are not limited to these embodiments.

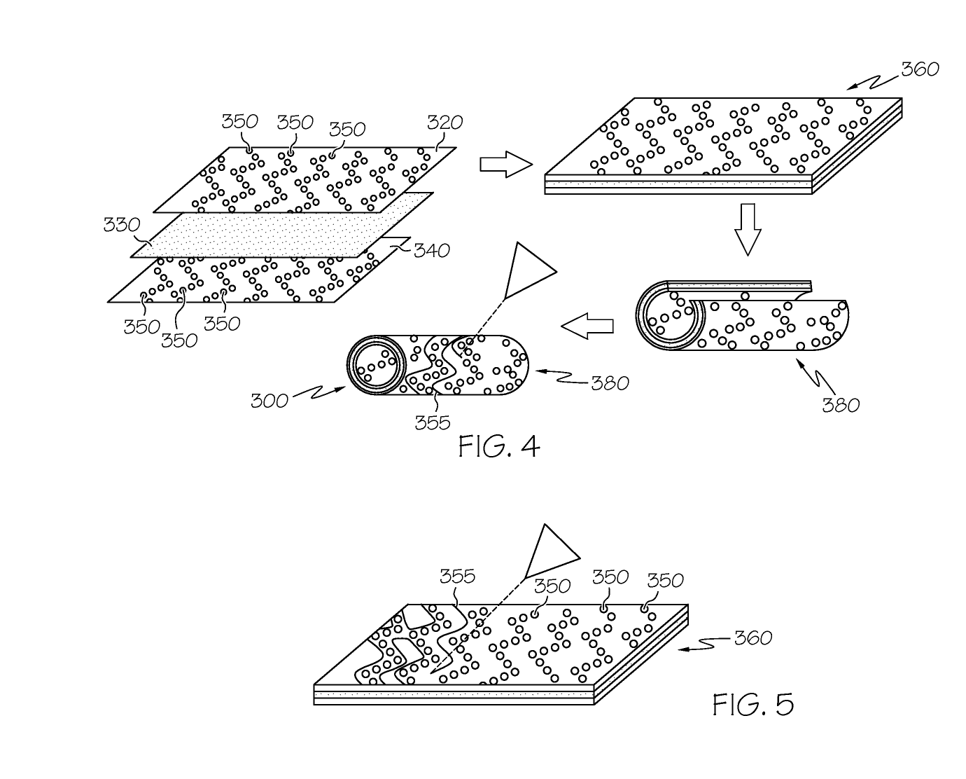

Referring to FIG. 4, one method to manufacture stent 300 is to cut holes, reservoirs, and openings 350 in the desired layer 320, 340. These openings 350 can be cut using methods common to current stent cutting technologies: laser, water jet, milling, etc. After cutting the holes 350 into the individual layer 320, 340, the layers can then be joined to make a single flat composite sheet 360. In order to join layers 320, 330, 340, various methods can be used depending on the types of materials being joined. These methods include but are not limited to brazing, adhesives, tie layers, metallurgically bonding, heat treating, annealing, wrapping, or fitting concentric tubes together and then drawing them through a sizing die. Once the materials are joined or otherwise bonded together, the composite sheet 360 can then be rolled into a tube-shaped form 380, and the ends welded or otherwise bonded. The final stent 300 can then be cut from the tube 380.

Referring now to FIG. 5, another method of manufacturing stent 300 would be to cut holes, reservoirs or openings 350 and a stent pattern 355 out of the flat composite sheet 360 prior to rolling sheet 360 into tube 380, and then roll the sheet 360 and weld into a tube 380. Stent pattern 355 consists of a combination of struts, connectors and other members which form the perimeter of a given cell. Reservoirs are defined by and thus essentially within a given strut or connector.

Another method of creating the composite sheet 360 involves depositing layers of material directly onto a first layer or a support layer. The material can be deposited using techniques such as those, described in U.S. Pat. No. 4,485,387 to Drumheller and incorporated herein by reference, to create the walls of the reservoirs for the first layer, or can be sequentially deposited using techniques such as vapor deposition. Sintering and/or molding can also be used to create multiple-layered composite tubes depending on the material. The composite tube can also be made by starting with a first layer shaped into a tube and filling the tube with another material. After that material solidifies, it can be bored out to create a tube of two materials. This process can be repeated again for tubes of more than two materials, again by either boring out or gun drilling out the center area in order to make a tubular structure.

Another embodiment would be to cut a first inner layer with drug reservoirs from a tube using conventional stent processing techniques as laser cutting. A support layer could then be wrapped around and joined to the first layer. A second layer could then be wrapped around and joined to the support layer. Preferably the second layer would be laser cut with drug reservoirs prior to bonding to the support layer, but if the second layer remained solid the reservoirs could be removed prior to the stent pattern being cut. Once the various layers are joined together the stent pattern could then be cut out.

Another method for manufacturing the device is to start with a wire or other tubular member of some material and then coat the wire or wrap the wire with another material to form a first layer. Once coated, reservoirs could be cut into the first outer layer. The wire or tubular member could then be shaped into stent undulation columns. The undulation columns could be joined together by welding, securing together with another material such as sutures, or attached by other methods such as adhesives.

In some embodiments the stent includes one or more areas, bands, coatings, members, etc. that is (are) detectable by imaging modalities such as X-Ray, MRI, ultrasound, etc. In some embodiments at least a portion of the stent and/or adjacent assembly is at least partially radiopaque. The imaging modality may be included into one or more of the various layers of the stent 100, such as the support layer, first layer, second layer, etc.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims.

This completes the description of the preferred and alternate embodiments of the invention. Those skilled in the art may recognize other equivalents to the specific embodiment described herein which equivalents are intended to be encompassed by the claims attached hereto.

1. An expandable tubular stent comprising:
a wall, the wall defining an inner stent surface and an outer stent surface and a thickness, the wall having a plurality of interconnected stent members, adjacent members defining openings, each opening extending entirely through the wall;
the wall comprising a support layer and a first layer engaged to the support layer;
the support layer and the first layer each having a thickness;
the support layer constructed from a support layer material and the first layer constructed from at least one first layer material, the at least one first layer material being different than the support layer material; and
the first layer having at least one reservoir, the at least one reservoir extending at least partially through the thickness of at least the first layer.

2. The stent of claim 1 wherein the first layer comprises a plurality of layers constructed from at least one first layer material.

3. The stent of claim 1 further comprising a second layer engaged with the support layer, the second layer constructed from at least one second layer material, the at least one second layer material being different than the support layer material.

4. The stent of claim 3 wherein the second layer comprises a plurality of layers constructed from at least one second layer material.

5. The stent of claim 3 wherein the second layer has at least one reservoir which extends at least partially through the thickness of at least the second layer.

6. The stent of claim 5 wherein the at least one reservoir of the first layer and the at least one reservoir of the second layer are substantially parallel.

7. The stent of claim 5 wherein the at least one reservoir of the second layer is offset from the at least one reservoir of the first layer.

8. The stent of claim 3 wherein at least one reservoir of the first layer extends through the entire thickness of the first layer, at least one reservoir of the second layer extends through the entire thickness of the second layer, and the support layer material is permeable.

9. The stent of claim 1 or claim 3 wherein at least one reservoir contains at least one therapeutic agent.

10. An expandable tubular stent comprising:
a wall, the wall defining an inner stent surface, an outer stent surface and a thickness, the wall having a plurality of interconnected stent members, adjacent members defining openings, each opening extending entirely through the wall;
the wall comprising the stent member covered with at least a first layer;
the stent member and the first layer each having a thickness;
the first layer constructed from a first layer material; and
the first layer having at least one reservoir, the at least one reservoir extending at least partially through the thickness of at least the first layer.

11. The stent of claim 10 wherein the at least one reservoir contains at least one therapeutic agent.

12. The stent of claim 10 wherein the first layer comprises a plurality of layers constructed from at least one first layer material.

13. A method of manufacturing an expandable stent comprising:
cutting at least one reservoir out of at least a first layer;
joining the first layer to at least a second layer to form a composite sheet;
rolling the composite sheet into a tube and joining the ends; and
cutting a stent pattern out of the composite sheet.

14. The method of claim 13 wherein the at least one reservoir is cut using at least one method of the group consisting of: laser cutting, water jet cutting, milling, drilling and any combination thereof.

15. The method of claim 13 wherein the second layer is made from a second layer material deposited onto the first layer to form the composite sheet.

16. The method of claim 13 wherein the first layer is a tubular-shaped layer and the second layer is joined to the first layer by wrapping the second layer around the first layer.

17. The method of claim 13 wherein the first layer and the second layer are concentric tubes that are joined together by drawing them through a sizing die.

18. A method of manufacturing an expandable stent comprising:
shaping a first layer into a tube from a first layer material, said tube having a lumen;
filling the lumen with at least a second layer material;
removing a first quantity of the second layer material to create at least a second layer and form a composite tube;
cutting at least one reservoir out of at least the first layer, said reservoirs extending at least partially through the first layer; and
cutting a stent pattern out of the composite tube.

19. A method of manufacturing an expandable stent comprising:
adhering a first layer to a wire member such that the first layer surrounds the wire member, said first layer being made from a first layer material;
forming at least one reservoir by removing a first quantity of the first layer material from the first layer;
shaping the wire member into stent undulation columns, said columns being joined together to form the wall of the stent.

20. The method of claim 19 wherein the first layer comprises a plurality of layers constructed from at least one first layer material.
